(19) United States
(12) Patent Application Publication
(10) Pub. No.: US 2022/0409067 A1
(43) Pub. Date: Dec. 29, 2022

(54) ARRAY TYPE SKIN-CONFORMAL SENSOR FOR HEART RATE AND BODY TEMPERATURE MEASUREMENT AND HEART RATE AND BODY TEMPERATURE MEASUREMENT APPARATUS

(71) Applicant: KOREA ELECTRONICS TECHNOLOGY INSTITUTE, Seongnam-si (KR)

(72) Inventor: Kunnyun KIM, Yongin-si (KR)

(21) Appl. No.: 17/704,182

(22) Filed: Mar. 25, 2022

(30) Foreign Application Priority Data
Jun. 28, 2021 (KR) 10-2021-0084219

(51) Int. Cl. A61B 5/0205; A61B 5/024; A61B 5/00

(52) U.S. Cl. CPC A61B 5/02055; A61B 5/02438; A61B 5/6833; A61B 2562/066; A61B 2562/18; A61B 2562/164

(57) ABSTRACT

Disclosed are an array type skin-conformal sensor for heart rate and body temperature measurement, the array type skin-conformal sensor including a base sheet configured to be attached to the skin in tight contact therewith, the base sheet being made of a skin-conformal material, a measurement portion formed at one surface of the base sheet, the measurement portion being deformed by deformation of the skin, whereby resistance of the measurement portion is changed, and an electrode pattern formed at one surface of the base sheet, one end of the electrode pattern being connected to the measurement portion to transmit a change in resistance of the measurement portion, and a heart rate and body temperature measurement apparatus using the same. Since the skin-conformal sensor is attached to the skin in tight contact therewith, it is possible to measure the heart rate even though a blood vessel is deeply located.

### CROSS REFERENCE TO RELATED APPLICATION

The present application claims priority to Korean Patent Application No. 10-2021-0084219, filed on Jun. 28, 2021, the entire contents of which is incorporated herein for all purposes by this reference.

### BACKGROUND OF THE INVENTION

### Field of the Invention

The present invention relates to an array type skin-conformal sensor for heart rate and body temperature measurement and a heart rate and body temperature measurement apparatus.

### Description of the Related Art

Heart rate varies between the maximum blood pressure when the heart contracts and the minimum blood pressure when the heart relaxes. Heart rate is one of the indices indicating a health state, and there are various methods of accurately measuring the heart rate. There are an optical type photoplethysmography (PPG) sensor and a pressure-sensor-based tonometry sensor as conventional heart rate sensors.

The PPG sensor measures a change in volume of an artery or a vein. When an LED of the PPG sensor irradiates light to a blood vessel under the skin, the light passes through the skin and is reflected by the blood vessel, and a photodiode measures the reflected light. The light emitted by the PPG sensor is transmitted through the skin to a limited depth, whereby the PPG sensor may be used for a superficial artery located immediately under the skin; however, it is difficult for the PPG sensor to be used for an artery located deeply under the skin, such as a coronary artery or a carotid artery. In addition, a larger amount of light is absorbed as subcutaneous fat is thicker, and therefore measurement may be difficult depending on people.

For the tonometry sensor, a measurer must press a measurement probe against the skin at the position at which the blood vessel is located with appropriate force. If the force with which the measurer presses the probe is too large or too small, a heart rate waveform may be distorted. In addition, when the measurer does not accurately dispose the probe on the blood vessel, it is difficult to measure the heart rate.

Meanwhile, in recent years, wearable devices capable of measuring heart rate, such as a wrist watch, have been popularized. A PPG sensor or a pressure sensor is disposed inside of a band of the wrist watch in order to measure the heart rate. However, it is difficult for the band of the wrist watch to be brought into tight contact with the skin, and it is difficult for the sensor to be maintained in a state of being accurately located on a radial artery in daily life, whereby it is difficult to accurately measure the heart rate.

### RELATED ART DOCUMENTS

### Patent Documents

(Patent Document 1) KR 10-2021-0045020 A

### SUMMARY OF THE INVENTION

It is an object of the present invention to provide a heart rate and body temperature measurement sensor capable of being attached to the skin using a skin-conformal material.

It is another object of the present invention to provide a heart rate and body temperature measurement sensor capable of stably acquiring the heart rate using measurement portions arranged in an array even though a user attaches the sensor to a position adjacent to the blood vessel but deviating therefrom.

In accordance with an aspect of the present invention, the above and other objects can be accomplished by the provision of an array type skin-conformal sensor for heart rate and body temperature measurement, the array type skin-conformal sensor including a base sheet configured to be attached to the skin in tight contact therewith, the base sheet being made of a skin-conformal material, a measurement portion formed at one surface of the base sheet, the measurement portion being deformed by deformation of the skin, whereby resistance of the measurement portion is changed, and an electrode pattern formed at the one surface of the base sheet, one end of the electrode pattern being connected to the measurement portion to transmit a change in resistance of the measurement portion.

The measurement portion may include a plurality of heart rate measurement portions formed at the one surface of the base sheet so as to be disposed in an array and at least one body temperature measurement portion formed at the one surface of the base sheet.

The array type skin-conformal sensor may further include a deformation prevention layer formed at the one surface or the other surface of the base sheet, the deformation prevention layer being made of a material having lower elasticity than the base sheet to limit elasticity of a part of the base sheet.

The deformation prevention layer may include a temperature measurement support portion formed at the portion at which the body temperature measurement portion is disposed such that deformation of the skin due to heart rate is not transmitted to the body temperature measurement portion.

The deformation prevention layer may include a sensor shape fixing portion formed at the edge of the base sheet to inhibit expansion of the base sheet to a damage limit or more.

The deformation prevention layer may include a shielding portion spaced apart from the measurement portion, the shielding portion being constituted by one or more parts so as to surround at least a part of the measurement portion, the shielding portion being configured to prevent deformation of the skin occurring due to a cause other than the heart rate from being transmitted to the measurement portion.

The array, in which the plurality of heart rate measurement portions is disposed, may be any one of a cross array, a matrix array, an oblique array, a circular array, and a decentralized array.

Each of the plurality of heart rate measurement portions may be formed in any one of a circular shape, a quadrangular shape, a diamond shape, a serpentine shape, and a linear shape.

In accordance with another aspect of the present invention, there is provided a heart rate and body temperature measurement apparatus including a skin-conformal sensor configured to be attached to the skin, the skin-conformal sensor being configured to convert expansion and contraction of the skin caused by heart rate into resistance change and to output the resistance change, the skin-conformal sensor also being configured to convert body temperature into resistance change and to output the resistance change, and a reader configured to analyze the resistance change received from the skin-conformal sensor and to calculate the heart rate and body temperature.

The skin-conformal sensor may include a base sheet configured to be attached to the skin in tight contact therewith, the base sheet being made of a skin-conformal material, a plurality of heart rate measurement portions formed at one surface of the base sheet so as to be disposed in an array, the plurality of heart rate measurement portions being deformed according to expansion and contraction of the skin to output resistance change, at least one body temperature measurement portion formed at the one surface of the base sheet, the body temperature measurement portion being configured to output resistance change depending on a change in body temperature, and an electrode pattern formed at the one surface of the base sheet, one end of the electrode pattern being connected to the heart rate measurement portions or the body temperature measurement portion to transmit resistance change of the heart rate measurement portions or the body temperature measurement portion.

The reader may include a reception unit configured to receive the resistance change from the plurality of heart rate measurement portions and the at least one body temperature measurement portion and to generate output signals over time, a heart rate calculation unit configured to analyze the output signals generated by the reception unit for the plurality of heart rate measurement portions and to calculate the heart rate from output signals having the same shape as a reference waveform, excluding output signals having different shapes from the reference waveform, and a body temperature calculation unit configured to analyze the output signal generated by the reception unit for the at least one body temperature measurement portion and to calculate a body temperature corresponding to a resistance value.

When one or more of the output signals generated for the plurality of heart rate measurement portions include an artery pulse, the heart rate calculation unit may average heart rates calculated from the output signals to calculate a final heart rate.

The heart rate calculation unit may compare an output signal including an artery pulse with an output signal including no artery pulse in real time, among waveforms corresponding to the plurality of heart rate measurement portions, when a peak having the same shape occurs at the same time, may determine the peak to be noise, and may exclude the peak from heart rate calculation.

The body temperature calculation unit may input the output signal of each heart rate measurement portion to a low pass filter in order to acquire a filtered output signal, from which an artery pulse component has been removed, and to calculate body temperature.

The features and advantages of the present invention will be more clearly understood from the following detailed description taken in conjunction with the accompanying drawings.

It should be understood that the terms used in the specification and appended claims should not be construed as being limited to general and dictionary meanings, but should be construed based on meanings and concepts according to the spirit of the present invention on the basis of the principle that the inventor is permitted to define appropriate terms for the best explanation.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a view showing the state in which an array type skin-conformal sensor for heart rate and body temperature measurement according to an embodiment of the present invention is attached to the wrist of a user;

FIG. 2 is a view showing the array type skin-conformal sensor for heart rate and body temperature measurement according to the embodiment of the present invention;

FIG. 3 is a sectional view of the skin-conformal sensor taken along line A-A′ of FIG. 2;

FIGS. 4A and 4B are a view showing skin-conformal contact and non-conformal contact;

FIGS. 5A and 5B are a view showing the state in which a skin-conformal sensor including a protective layer according to an embodiment of the present invention is attached to the skin and an output signal at that time;

FIGS. 6A and 6B is a view showing the state in which a skin-conformal sensor including an upper attachment layer according to an embodiment of the present invention is attached to the skin and an output signal at that time;

FIGS. 7A and 7B is a view showing the state in which the skin-conformal sensor according to the embodiment of the present invention is attached to the skin using only a base sheet and an output signal at that time;

FIG. 8 is a view showing the difference between the skin-conformal sensor according to the embodiment of the present invention and a conventional strain gauge type sensor;

FIGS. 9A, 9B, and 9C are a view showing attachment directions of the array type skin-conformal sensor for heart rate and body temperature measurement according to the embodiment of the present invention;

FIG. 10 is a view showing the array type skin-conformal sensor for heart rate and body temperature measurement according to the embodiment of the present invention and pulse waveforms depending on the position of a blood vessel;

FIG. 11 is a view showing a heart rate and body temperature measurement apparatus including a skin-conformal sensor and a reader according to an embodiment of the present invention;

FIG. 12 is a view showing waveforms output respectively by measurement portions disposed in a cross array according to an embodiment of the present invention;

FIG. 13 is a graph showing an output signal of a body temperature measurement portion of the skin-conformal sensor;

FIG. 14 is a view showing a process in which a body temperature calculation unit according to an embodiment of the present invention calculates body temperature using an output signal of a heart rate measurement portion;

FIG. 15 is a view showing that the skin-conformal sensor according to the embodiment of the present invention is separated from the skin;

FIG. 16 is a view showing a skin-conformal sensor further including a sensor shape fixing portion according to an embodiment of the present invention; and

FIG. 17 is a view showing a skin-conformal sensor further including a shielding portion according to an embodiment of the present invention.

### DETAILED DESCRIPTION OF THE INVENTION

Objects, specific advantages, and novel features of an embodiment of the present invention will be apparent from exemplary embodiments and the following detailed description in connection with the accompanying drawings. It should be noted that when reference numerals are assigned to the elements of the drawings, the same reference numeral is assigned to the same elements even when they are illustrated in different drawings. Furthermore, the terms “one surface”, “the other surface”, “first”, “second”, etc. are only used to distinguish one element from another element, and these elements are not to be construed as being limited by these terms. In the following description of an embodiment of the present invention, a detailed description of known technology incorporated herein will be omitted when it may obscure the subject matter of an embodiment of the present invention.

Hereinafter, an embodiment of the present invention will be described in detail with reference to the accompanying drawings.

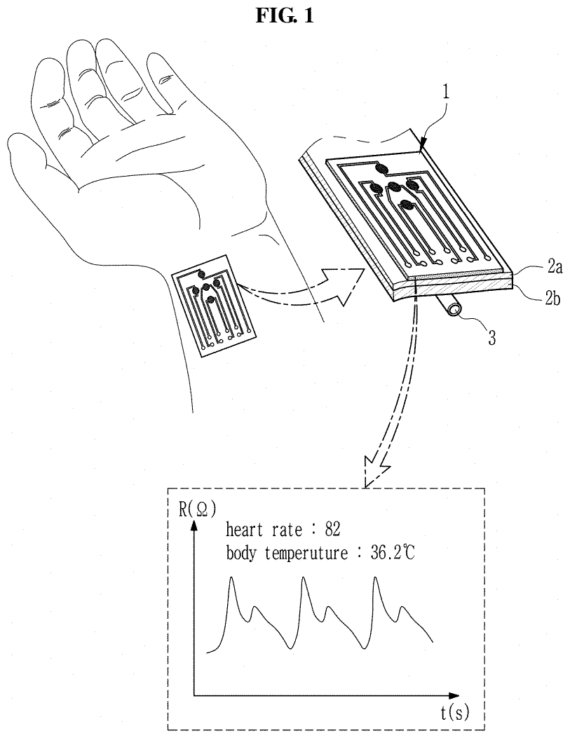

FIG. 1 is a view showing the state in which an array type skin-conformal sensor 1 for heart rate and body temperature measurement according to an embodiment of the present invention is attached to the wrist of a user. In this specification, the “array type skin-conformal sensor 1 for heart rate and body temperature measurement” may simply be referred to as a “skin-conformal sensor 1.”

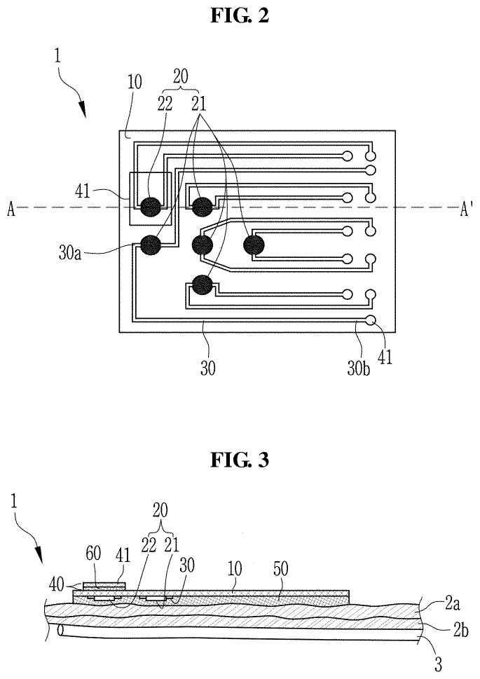

FIG. 2 is a view showing the array type skin-conformal sensor 1 for heart rate and body temperature measurement according to the embodiment of the present invention. FIG. 3 is a sectional view of the skin-conformal sensor 1 taken along line A-A′ of FIG. 2. FIGS. 1, 2, and 3 are referred to throughout this specification.

The array type skin-conformal sensor 1 for heart rate and body temperature measurement according to the embodiment of the present invention may include a base sheet 10 configured to be attached to the skin 2a in tight contact therewith, the base sheet being made of a skin-conformal material, a measurement portion 20 formed at one surface of the base sheet 10, the measurement portion being deformed by deformation of the skin 2a, whereby resistance of the measurement portion is changed, and an electrode pattern 30 formed at one surface of the base sheet 10, one end of the electrode pattern being connected to the measurement portion 20 in order to transmit a change in resistance of the measurement portion.

The base sheet 10 may be made of a skin-conformal and skin-compatible material. The base sheet 10 may be made of a skin-conformal material that has similar physical properties to the skin 2a. The base sheet 10 may have elasticity similar to or higher than the elasticity of the skin 2a. Consequently, the base sheet 10 may be attached to the skin 2a in tight contact therewith. The base sheet 10 may be made of a skin-compatible material that is safe even when directly attached to the skin 2a. When being attached to the skin 2a, the base sheet 10 may also be deformed according to deformation of the skin 2a.

Since the base sheet 10 has similar physical properties to the skin 2a, the base sheet 10 may expand according to the movement of a blood vessel 3, although the blood vessel 3 is under the skin 2a and a fat layer 2b. Even in the case in which the fat layer 2b is thick or the blood vessel 3 is located deeply from the skin 2a, the base sheet 10 may be deformed even by a microscopic change of the skin 2a due to the movement of the blood vessel 3, since elasticity of the base sheet 10 is higher than elasticity of the skin 2a.

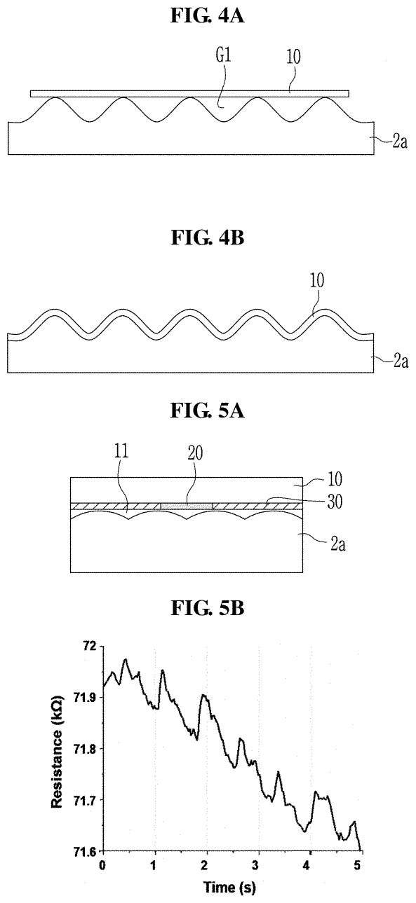

FIGS. 4A and 4B are a view showing skin-conformal contact and non-conformal contact. The surface of the skin 2a is not smooth but is irregular. In the case in which the base sheet 10 is made of a non-conformal material, a gap G1 may be formed between the skin 2a and the base sheet 10 when the base sheet 10 is attached to the skin 2a, as shown in FIG. 4A. When the gap G1 is formed between the skin 2a and the base sheet 10, the base sheet 10 has difficulty receiving deformation of the skin 2a even though the skin 2a contracts or expands. That is, it is difficult for the base sheet 10 to receive deformation of the skin 2a caused by the heart rate, whereby sensitivity of the sensor is low.

In the case in which the base sheet 10 is made of a skin-conformal material, the base sheet 10 is attached along the irregularities of the skin 2a, whereby no gap G1 is formed therebetween, when the base sheet 10 is attached to the skin 2a, as shown in FIG. 4B. When the base sheet 10 is attached along the irregularities of the skin 2a, the base sheet 10 also contracts or expands when the skin 2a contracts or expands. Consequently, it is possible for the base sheet 10 to completely receive deformation of the skin 2a caused by the heart rate, whereby sensitivity of the sensor is high. A skin-conformal material suitable for the base sheet 10 is a material having a lower Young's modulus than the skin 2a. Table 1 below shows materials or products (with a superscript of TM) from which a sheet that can be attached to the skin is capable of being manufactured and Young's moduli thereof for comparison with the skin and Young's modulus thereof.

TABLE 1

| No. | Material | Young's modulus (Pa) | |
|---|---|---|---|
| 1 | Polyimide (PI) | 2.8 | GPa |
| 2 | Polydimethylsiloxane (PDMS) | 3.7 | MPa |
| 3 | Solaris | 251.53 | kPa |
| 4 | Polyurethane (PU) | 28.13 | kPa |
| 5 | Clear Flex 30 ™ | 15.76 | kPa |
| 6 | Skin (Wrist) | 10.45 | kPa |
| 7 | Tegaderm ™ | 4.75 | kPa |
| 8 | Ecoflex 0030 ™ | 1.53 | kPa |

Materials having smaller Young's moduli than the skin 2a are skin-conformal materials. For example, Tegaderm™ and Ecoflex 0030™, which are materials having smaller Young's moduli than the skin 2a, are skin-conformal materials. The base sheet may be made of an elastomer, Tegaderm™ or Ecoflex 0030™. Since the Young's modulus of the skin 2a varies depending on positions of the body, Clear Flex 30™, which has slightly higher Young's modulus than the skin 2a of the wrist, may be a skin-conformal material when a skin-conformal sensor 1 attached to the skin 2a of other parts of the body is manufactured. That is, a skin-conformal material is a material having lower Young's modulus than the skin 2a of the part of the body to which the skin-conformal sensor 1 is attached. A composite material may be used as the base sheet 10. Consequently, the base sheet 10 may be made of a material including polyurethane (PU), polyimide (PI), polydimethylsiloxane (PDMS), and Solaris, which are combined so as to have lower Young's modulus than the skin 2a.

The measurement portion 20 is formed at one surface of the base sheet 10. The measurement portion 20 may include a sensing material, such as carbon nanotube (CNT), carbon, graphite, graphene, metal particles, metal nanopowder, and conductive powder. The measurement portion 20 may further include a binder in order to fix components of the sensing material to each other and to fix the sensing material to the base sheet. The measurement portion 20 may be formed by applying the sensing material to one surface of the base sheet 10 so as to have a predetermined size, shape, and thickness. The measurement portion 20 may be formed at one surface of the base sheet 10 by screen printing. FIG. 3 shows the case in which the measurement portion 20 is formed at one surface of the base sheet 10 that contacts the skin 2a. The measurement portion 20 may be formed at the other surface of the base sheet 10 that does not contact the skin 2a. When the base sheet 10 is deformed according to deformation of the skin 2a, the measurement portion 20 may be deformed according to deformation of the base sheet 10. When the measurement portion 20 is deformed, an electrical resistance value of the measurement portion 20 is changed.

The electrode pattern 30 may be formed at one surface of the base sheet 10 to transmit an electrical signal. One end 30a of the electrode pattern 30 may be connected to the measurement portion 20, and the other end 30b of the electrode pattern 30 may be connected to an electrode pad 31 formed at the base sheet 10. The electrode pad 31 may be connected to an external circuit. The electrode pad 31 may be formed at one surface or opposite surfaces of the base sheet 10 so as to be easily connected to a connector 6. The electrode pattern 30 may be connected to each of opposite ends of the measurement portion 20.

The skin-conformal sensor 1 may further include a protective layer 11 configured to cover the electrode pattern 30 and the measurement portion 20 in order to protect the electrode pattern 30 and the measurement portion 20, the protective layer 11 being attached to the skin. The protective layer 11 may be made of a material that is adhesive so as to be easily attached to the skin 2a. The protective layer 11 may be formed so as to be much thinner than the base sheet 10 in order to maximally transmit deformation of the skin 2a to the base sheet 10.

Sensitivity of the skin-conformal sensor 1 depending on presence or absence of the protective layer 11 and an upper attachment layer 12 will be described with reference to FIGS. 5A, 5B, 6A, 6B, 7A and 7B.

FIGS. 5A and 5B are a view showing the state in which a skin-conformal sensor 1 including a protective layer 11 according to an embodiment of the present invention is attached to the skin 2a and an output signal at that time.

As shown in FIG. 5A, the measurement portion 20 and the electrode pattern 30 may be formed at one surface of the base sheet 10, a protective layer 11 configured to cover the measurement portion 20 and the electrode pattern 30 may be formed at one surface of the base sheet 10, and the protective layer 11 may be attached to the skin 2a. In the skin-conformal sensor 1, the protective layer 11 is formed so as to have a thickness of 85 μm, and the base sheet 10 is made of Ecoflex™ so as to have a thickness of 0.5 mm At this time, the protective layer 11 may be made of an adhesive material, and may be attached to the skin 2a in tight contact with the irregularities of the skin 2a.

As shown in FIG. 5B, a percentage of the ratio of resistance variation ΔR to the resistance value Ro of a pulse included in an output signal, (ΔR /R₀)*100, is about 0.107%.

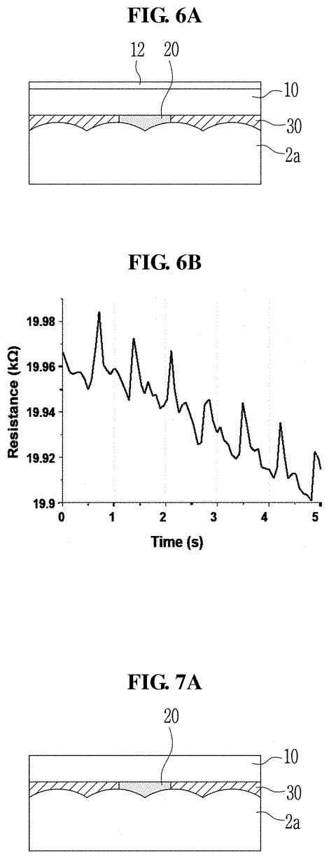

FIGS. 6A and 6B are a view showing the state in which a skin-conformal sensor 1 including an upper attachment layer 12 according to an embodiment of the present invention is attached to the skin 2a and an output signal at that time.

As shown in FIG. 6A, the measurement portion 20 and the electrode pattern 30 may be formed at one surface of the base sheet 10, the measurement portion 20 and the electrode pattern 30 may be attached so as to directly contact the skin 2a, an upper attachment layer 12 having a larger area than the base sheet 10 may be further formed at the other surface of the base sheet 10, and the portion of the upper attachment layer 12 that protrudes outwards from the base sheet 10 may be attached to the skin 2a. In the skin-conformal sensor 1, the upper attachment layer 12 is formed so as to have a thickness of 80 μm, and the base sheet 10 is made of Ecoflex™ so as to have a thickness of 0.5 mm Since no protective layer 11 is provided, unlike FIG. 5A, the base sheet 10 may be attached to the skin 2a in tight contact with the irregularities of the skin 2a.

As shown in FIG. 6B, a percentage of the ratio of resistance variation ΔR to the resistance value Ro of a pulse included in an output signal, (ΔR /R₀)*100, is about 0.139%.

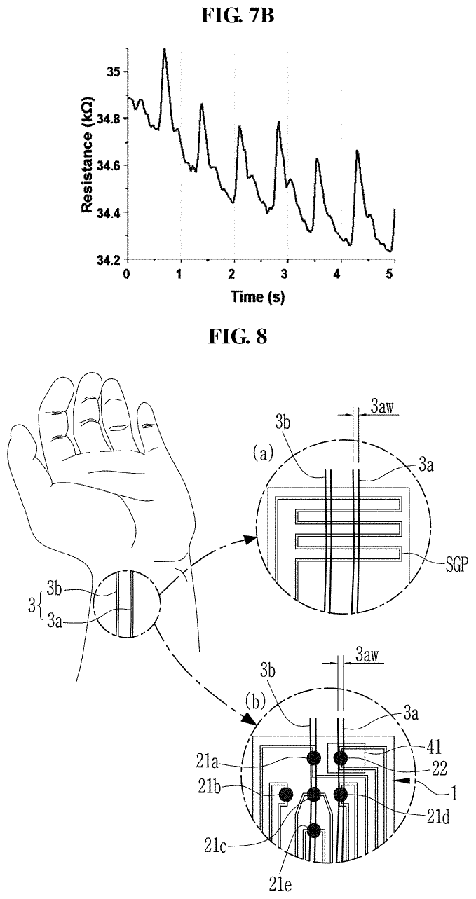

FIGS. 7A and 7B are a view showing the state in which the skin-conformal sensor 1 according to the embodiment of the present invention is attached to the skin 2a using only the base sheet 10 and an output signal at that time.

As shown in FIG. 7A, the measurement portion 20 and the electrode pattern 30 may be formed at one surface of the base sheet 10, the measurement portion 20 and the electrode pattern 30 may be attached so as to directly contact the skin 2a, and neither the protective layer 11 nor the upper attachment layer 12 is provided. In the skin-conformal sensor 1, the base sheet 10 is made of Ecoflex™ so as to have a thickness of 0.5 mm Since no protective layer 11 is provided, unlike FIG. 5A, the base sheet 10 may be attached to the skin 2a in tight contact with the irregularities of the skin 2a. The state in which the base sheet 10 is attached to the skin 2a may be maintained, since the base sheet 10 is made of a skin-conformal material, even though no upper attachment layer 12 is provided, unlike FIG. 6A.

As shown in FIG. 7B, a percentage of the ratio of resistance variation ΔR to the resistance value R₀ of a pulse included in an output signal, (ΔR/R₀)*100, is about 0.954%.

As described above with reference to FIGS. 5A, 5B, 6A, 6B, 7A and 7B, the skin-conformal sensor 1 may be attached to the skin 2a in tight contact therewith using only the base sheet 10, since the base sheet 10 is made of a skin-conformal material. When an adhesive layer, such as a protective layer 11, is not provided between the base sheet 10 and the skin 2a and an additional layer configured to cover the base sheet 10, such as an upper attachment layer 12, is not provided, it can be seen that the ratio of resistance variation to the resistance value is the greatest.

The measurement portion 20 may include a plurality of heart rate measurement portions 21 formed at one surface of the base sheet 10 so as to be disposed in an array and one or more body temperature measurement portions 22 formed at one surface of the base sheet 10. The heart rate measurement portions 21 and the body temperature measurement portions 22 may be made of the same material so as to have the same shape. Portions that analyze a change in resistance to measure the heart rate from an output signal of the measurement portion 20 may be the heart rate measurement portions 21, and portions that analyze a change in resistance to measure the body temperature may be the body temperature measurement portions 22.

The measurement portion 20 may be formed in any one of a circular shape, a quadrangular shape, a diamond shape, a serpentine shape, and a linear shape. The heart rate measurement portions 21 and the body temperature measurement portions 22 may be formed in different shapes. In the drawings of the present application, the measurement portion 20 is shown as having a circular shape; however, the present invention is not limited thereto.

FIG. 8 is a view showing the difference between the skin-conformal sensor 1 according to the embodiment of the present invention and a conventional strain gauge type sensor.

The blood vessel 3 includes an artery 3a and a vein 3b. In general, the heart rate is calculated by measuring the flow of blood in the artery 3a. A conventional heart rate measurement sensor using a single sensor has difficulty in accurate measurement of the heart rate unless a user accurately disposes the sensor at the position of the blood vessel 3 (the artery 3a or the vein 3b). Since the blood vessel 3 is located under the skin 2a, it is difficult for the user to accurately dispose the sensor at the position of the blood vessel 3.

In order to solve a problem in that it is difficult for the user to accurately dispose the sensor at the position of the blood vessel 3, a comparative sensor Ct-1 having a wide measurement area may be provided. The comparative sensor Ct-1 is shown in (a) of FIG. 8, which is an enlarged view. The comparative sensor Ct-1 is of a strain gauge type. A strain gauge pattern SGP of the comparative sensor Ct-1 may be formed so as to be wider than the width 3aw of the artery 3a. Even when the center of the comparative sensor Ct-1 is not accurately located on the artery 3a, it is possible to measure the flow of blood flowing in the artery 3a, since a part of the strain gauge pattern SGP covers the artery 3a.

However, the wide strain gauge pattern SGP may also be attached so as to cover the vein 3b located adjacent to the artery 3a. In this case, the comparative sensor Ct-1 may measure the flow of blood flowing in the vein 3b. As a result, an output signal is distorted, and the heart rate is not accurately measured. The skin-conformal sensor 1 according to the embodiment of the present invention is shown in (b) of FIG. 8, which is an enlarged view. A plurality of measurement portions 20 may be disposed in an array. The array may be any one of a cross array, an m x n matrix array, an oblique array, a circular array, and a decentralized array. The plurality of measurement portions 20 may be disposed so as to be spaced apart from each other by a predetermined distance. The enlarged view of (b) of FIG. 8 shows the skin-conformal sensor 1 having a structure in which the plurality of measurement portions 20 is disposed in a cross array; however, the present invention is not limited thereto.

In the skin-conformal sensor 1 according to the embodiment of the present invention, at least one of the plurality of measurement portions 20 is disposed adjacent to the artery 3a even though the user attaches the skin-conformal sensor 1 so as to slightly deviate from the artery 3a, since the plurality of measurement portions 20 is disposed in an array. Consequently, it is possible to stably measure the heart rate.

Each measurement portion 20 of the skin-conformal sensor 1 may be formed so as to has a size (width or length) corresponding to the width of the blood vessel 3 (the artery 3a or the vein 3b). As shown in the enlarged view of (b) of FIG. 8, the size of the measurement portion 20 may be formed so as to be similar to the width 3aw of the artery 3a. Alternatively, the size of the measurement portion 20 may be formed so as to be less than the width of the blood vessel 3. When the size of the measurement portion 20 is formed so as to be similar to or less than the width of the blood vessel 3, one measurement portion 20 may measure only the flow of blood flowing in one blood vessel 3. In other words, the flow of blood flowing in two blood vessels 3 (the artery 3a and the vein 3b) located adjacent to one measurement portion 20 may not be measured. The measurement portion 20 located adjacent to the artery 3a may not measure the flow of blood flowing in the vein 3b, whereby an output signal may not be distorted.

The measurement portion 20 may have various sizes so as to correspond to the width of the blood vessel 3 at the position at which the skin-conformal sensor 1 will be attached. For example, the measurement portion 20 of the skin-conformal sensor 1 that is attached to the neck may be formed so as to have a size corresponding to the width of the carotid artery of the neck, and the measurement portion 20 of the skin-conformal sensor 1 that is attached to the wrist may be formed so as to have a size corresponding to the width of the radial artery of the wrist.

In the skin-conformal sensor 1, a plurality of measurement portions 20, each of which has a size corresponding to the width of the blood vessel 3, is formed in an array. When any one of the plurality of measurement portions 20 is located adj acent to the artery 3a, another measurement portion may be located adjacent to the vein 3b. An output signal of the measurement portion 20 located adjacent to the artery 3a may be used for heart rate measurement, and an output signal of the measurement portion 20 located adjacent to the vein 3b may not be used for heart rate measurement.

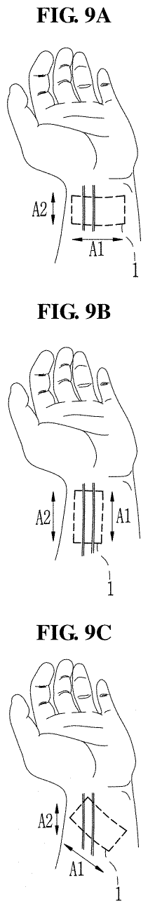

FIGS. 9A, 9B, and 9C are a view showing attachment directions of the array type skin-conformal sensor 1 for heart rate and body temperature measurement according to the embodiment of the present invention.

The skin-conformal sensor 1 may be attached in the state in which a longitudinal direction Al of the skin-conformal sensor 1 is perpendicular to an extension direction A2 of the blood vessel 3, as shown in FIG. 9A, may be attached in the state in which the longitudinal direction Al of the skin-conformal sensor 1 is parallel to the extension direction A2 of the blood vessel 3, as shown in FIG. 9B, or may be attached in the state in which the longitudinal direction Al of the skin-conformal sensor 1 is oblique to the extension direction A2 of the blood vessel 3, as shown in FIG. 9C. Even when the skin-conformal sensor 1 is attached at any angle with respect to the blood vessel 3, any one of the plurality of measurement portions 20 may be disposed on the artery 3a to measure the heart rate, since the plurality of measurement portions 20 is formed in an array.

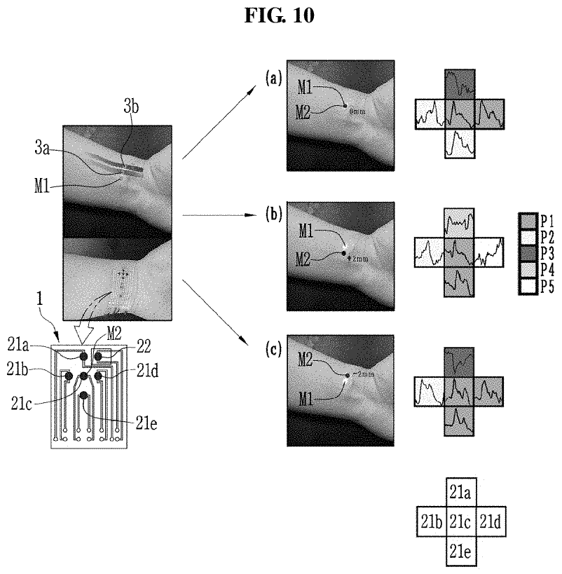

FIG. 10 is a view showing the array type skin-conformal sensor 1 for heart rate and body temperature measurement according to the embodiment of the present invention and pulse waveforms depending on the position of the blood vessel 3. The case in which the skin-conformal sensor 1 is attached to the wrist to measure the heart rate from the radial artery 3a will be described with further reference to FIG. 10. FIG. 10 shows a pulse waveform measured in the state in which the longitudinal direction Al of the skin-conformal sensor 1 is generally perpendicular to the extension direction A2 of the blood vessel 3 (see FIG. 9A).

A plurality of measurement portions 20 disposed in a cross array may be a first heart rate measurement portion 21a, a second heart rate measurement portion 21b, a third heart rate measurement portion 21c, a fourth heart rate measurement portion 21d, and a fifth heart rate measurement portion 21e in order from above. Pulse waveforms included in output signals of the heart rate measurement portions are shown in a cross lattice.

The position at which the skin-conformal sensor 1 is attached such that the middle M2 of the skin-conformal sensor 1 coincides with the middle M1 of the artery 3a is defined as a correct position. The middle M2 of the skin-conformal sensor 1 may be defined as the position of the third heart rate measurement portion 21c disposed at the center of the cross array. (a) of FIG. 10 shows the case in which the skin-conformal sensor 1 is disposed at the correct position, (b) of FIG. 10 shows the case in which the middle M2 of the skin-conformal sensor 1 deviates from the middle M1 of the artery 3a in a direction opposite the vein 3b, and (c) of FIG. 10 shows the case in which the middle M2 of the skin-conformal sensor 1 deviates from the middle M1 of the artery 3a in a direction toward the vein 3b.

When the skin-conformal sensor 1 is disposed at the correct position, as shown in (a) of FIG. 10, the first heart rate measurement portion 21a outputs a typical vein pulse P3, the second heart rate measurement portion 21b and the fifth heart rate measurement portion 21e output a non-typical artery pulse P2, and the third heart rate measurement portion 21c and the fourth heart rate measurement portion 21d output a typical artery pulse P1.

When the middle M2 of the skin-conformal sensor 1 deviates from the middle M1 of the artery 3a in a direction opposite the vein 3b, as shown in (b) of FIG. 10, the first heart rate measurement portion 21a outputs a non-typical vein pulse P4, the second heart rate measurement portion 21b outputs a non-typical artery pulse P2, the third heart rate measurement portion 21c and the fifth heart rate measurement portion 21e output a typical artery pulse P1, and the fourth heart rate measurement portion 21d outputs an ambiguous pulse P5, which is neither an artery pulse nor a vein pulse.

When the middle M2 of the skin-conformal sensor 1 deviates from the middle

M1 of the artery 3a in a direction toward the vein 3b, as shown in (c) of FIG. 10, the first heart rate measurement portion 21a outputs a typical vein pulse P3, the second heart rate measurement portion 21b outputs a non-typical artery pulse P2, and the third heart rate measurement portion 21c, the fourth heart rate measurement portion 21d, and the fifth heart rate measurement portion 21e output a typical artery pulse P1.

Since the plurality of heart rate measurement portions 21a to 21e of the skin-conformal sensor 1 is arranged in an array, at least one of the plurality of measurement portions 20 may be disposed at the position at which the typical artery pulse P1 is output even though the user does not accurately align the measurement portions 20 with the blood vessel 3. When the user attaches the skin-conformal sensor 1 to an approximate position through which the blood vessel 3 extends, therefore, any one of the plurality of measurement portions 20 outputs an artery pulse, whereby user convenience may be improved.

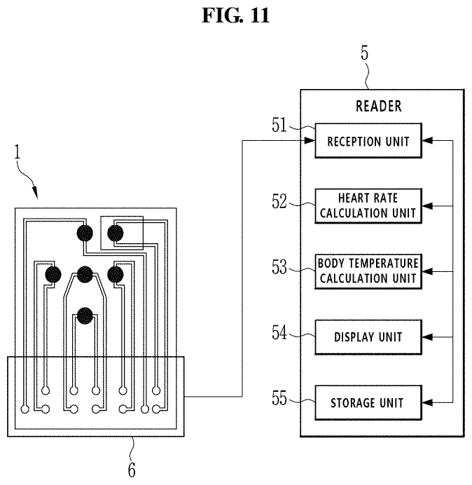

FIG. 11 is a view showing a heart rate and body temperature measurement apparatus including a skin-conformal sensor 1 and a reader 5 according to an embodiment of the present invention.

The heart rate and body temperature measurement apparatus according to the embodiment of the present invention may include a skin-conformal sensor 1 configured to be attached to the skin 2a, the skin-conformal sensor being configured to convert expansion and contraction of the skin 2a caused by the heart rate into resistance change and to output the resistance change, the skin-conformal sensor also being configured to convert body temperature into resistance change and to output the resistance change, and a reader 5 configured to analyze the resistance change received from the skin-conformal sensor 1 and to calculate the heart rate and the body temperature.

As described above, the skin-conformal sensor 1 may include a base sheet 10 configured to be attached to the skin 2a in tight contact therewith, the base sheet being made of a skin-conformal material, a plurality of heart rate measurement portions 21 formed in an array at one surface of the base sheet 10, the heart rate measurement portions being deformed according to expansion and contraction of the skin 2a to output resistance change, one or more body temperature measurement portions 22 formed at one surface of the base sheet 10, the body temperature measurement portions being configured to output resistance change depending on a change in body temperature, and an electrode pattern 30 formed at one surface of the base sheet 10, one end of the electrode pattern being connected to the heart rate measurement portions 21 or the body temperature measurement portions 22 in order to transmit the resistance change of the heart rate measurement portions 21 or the body temperature measurement portions 22.

The reader 5 may include a reception unit 51 configured to receive the resistance change from the plurality of heart rate measurement portions 21 and the one or more body temperature measurement portions 22 and to generate output signals over time, a heart rate calculation unit 52 configured to analyze the output signals generated by the reception unit 51 for the plurality of heart rate measurement portions 21 and to calculate the heart rate from output signals having the same shape as a reference waveform, excluding output signals having different shapes from the reference waveform, and a body temperature calculation unit 53 configured to analyze the output signals generated by the reception unit 51 for the one or more body temperature measurement portions 22 and to calculate a body temperature corresponding to a resistance value.

The reader 5 may further include a display unit 54 configured to display the heart rate and the body temperature, and a storage unit 55 configured to store the measured heart rate or body temperature over time and to store program code necessary to operate the reader 5. The reader 5 may further include an input unit (not shown) including a touchpad and a button configured to allow a user on/off signal or various commands to be input therethrough. The reader 5 may be a computing device that performs an information processing function. The reader 5 may be implemented in the form of a portable electronic device, a smartphone, a PC, a patient monitoring device, or a smart watch.

The skin-conformal sensor 1 may be connected to the reader 5 via a connector 6. The connector 6 may be connected to an electrode pad 31 of the skin-conformal sensor 1, and may also be connected to the reception unit 51 of the reader 5. The skin-conformal sensor 1 may be directly connected to the reception unit 51 of the reader 5. The reception unit 51 may record the resistance change of the measurement portion 20 over time to generate an output signal, which may be stored in the storage unit 55. The reception unit 51 may include a MUX, an AMP, an A/D converter, and a filter in order to convert an analog signal into a digital signal. The reception unit 51 may measure resistance change for each of the measurement portions 20, and may generate an output signal for each of the measurement portions 20, which may be stored in the storage unit 55.

The display unit 54 may visually or aurally output the heart rate calculated by the heart rate calculation unit 52, the waveform of the heart rate, and the body temperature calculated by the body temperature calculation unit 53. For example, as in the graph shown in FIG. 1, the heart rate (82), the body temperature (36.2), and the waveform of the heart rate may be displayed as a graph. The display unit 54 may include various displays, such as a touchscreen, and may also include a speaker. The user may check the heart rate and the body temperature visually displayed on the display unit 54.

The heart rate calculation unit 52 analyzes the output signal generated by the reception unit 51 to calculate the heart rate. The heart rate calculation unit 52 may analyze the output signal generated for each of the measurement portions 20 to calculate the heart rate for each of the measurement portions 20. The heart rate calculation unit 52 compares the output signal with a reference waveform. Here, the reference waveform is a reference artery pulse. The reference artery pulse has the form of a typical artery pulse. The waveform of the output signal is compared with the reference artery pulse to determine which output signal includes the artery pulse. The output signal including the same waveform as the reference artery pulse is used for heart rate calculation. The output signal including no artery pulse is not used for heart rate calculation.

When one or more of the output signals generated for the plurality of heart rate measurement portions 21 include an artery pulse, the heart rate calculation unit 52 may average heart rates calculated from the output signals to calculate the final heart rate.

A plurality of the output signals generated for the plurality of measurement portions 20 may include an artery pulse. That is, a plurality of heart rate measurement portions 21 may be accurately located on the artery 3a. In this case, the heart rate calculation unit 52 may randomly select any one of the plurality of output signals including the artery pulse to measure the heart rate, or may calculate the heart rates for the plurality of measurement portions 20 that accurately measure the artery pulse and may average the heart rates to calculate the final heart rate.

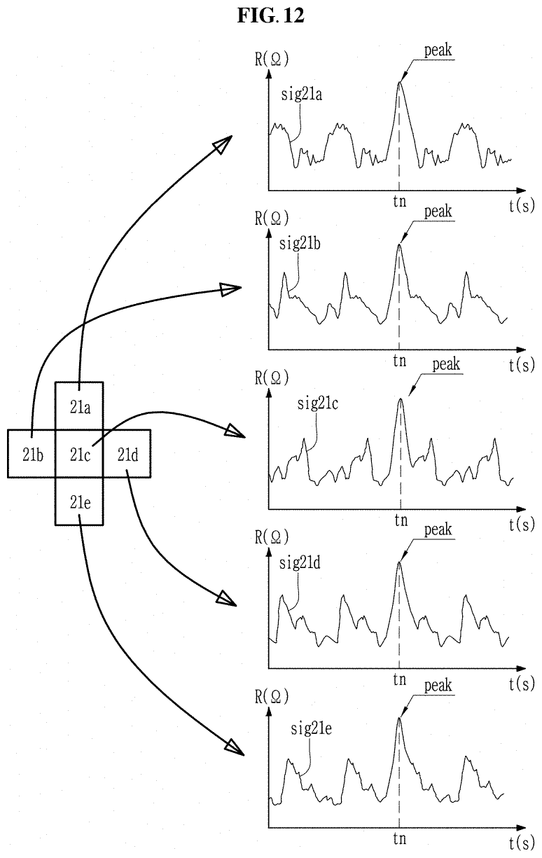

FIG. 12 is a view showing waveforms output respectively by measurement portions 20 disposed in a cross array according to an embodiment of the present invention.

The heart rate calculation unit 52 may compare an output signal including an artery pulse with an output signal including no artery pulse in real time, among waveforms corresponding to the plurality of heart rate measurement portions 21, when a peak having the same shape occurs at the same time, may determine the peak to be noise, and may exclude the peak from heart rate calculation.

FIG. 12 sequentially shows an output signal of the first heart rate measurement portion 21a, an output signal of the second heart rate measurement portion 21b, an output signal of the third heart rate measurement portion 21c, an output signal of the fourth heart rate measurement portion 21d, and an output signal of the fifth heart rate measurement portion 21e. The output signals including the artery pulse are the output signal of the third heart rate measurement portion 21c and the output signal of the fourth heart rate measurement portion 21d, and the output signals including no artery pulse are the output signal of the first heart rate measurement portion 21a, the output signal of the second heart rate measurement portion 21b, and the output signal of the fifth heart rate measurement portion 21e.

The skin 2a may temporarily expand or may be temporarily deformed due to user motion or user collision with an external object. Such deformation of the skin 2a is not deformation due to the heart rate, and thus must be excluded from heart rate measurement as noise. There is a possibility of deformation of the skin 2a due to a cause other than the heart rate generally occurring at the entirety of the portion at which the skin-conformal sensor 1 is attached. Consequently, noise may simultaneously cause deformation of the plurality of measurement portions 20.

When the same waveform (e.g. peak) is sensed at the same time to from the output signals of the plurality of measurement portions 20, the heart rate calculation unit 52 may determine the waveform to be noise and exclude the same from heart rate calculation. Some of the plurality of measurement portions 20 may include an artery pulse having the flow of blood in the artery 3a due to the heart rate reflected therein, and some other of the plurality of measurement portions 20 may include a vein pulse having the flow of blood in the vein 3b reflected therein or may include a waveform other than the artery pulse or the vein pulse. Consequently, the heart rate measurement portion 21 may determine a peak occurring in the output signal including the artery pulse and the output signal including no artery pulse to be noise. The peak determined to be noise may include both a positive peak having an increasing resistance value and a negative peak having a decreasing resistance value.

The body temperature calculation unit 53 may convert the resistance value of the measurement portion 20 into temperature. The resistance value of the measurement portion 20 is changed depending on temperature. A change in the resistance value depending on temperature is a value set during manufacture of the measurement portion 20. In general, a metal has a temperature-resistance proportional relationship, in which the resistance value thereof is increased when the temperature thereof is increased, and a semiconductor, a carbon-based material, or an insulating material has a temperature-resistance inversely proportional relationship, in which the resistance value thereof is decreased when the temperature thereof is increased. The measurement portion 20 may be set to have a temperature-resistance proportional relationship or a temperature-resistance inversely proportional relationship depending on a combination of a sensing material and a binder. A change in the resistance value of the measurement portion 20 depending on temperature change may be stored in the storage unit 55 as a mathematical expression, or may be stored in the form of a temperature-resistance value matching table.

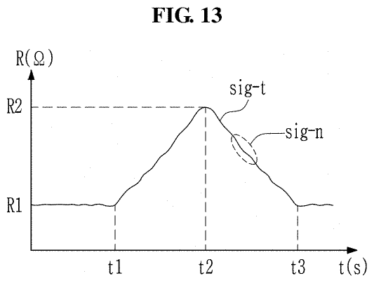

FIG. 13 is a graph showing an output signal sig-t of the body temperature measurement portion 22 of the skin-conformal sensor 1. FIG. 13 shows resistance change depending on an increase and decrease in body temperature when the body temperature measurement portion 22 has a temperature-resistance proportional relationship.

In FIG. 13, the portion of the skin 2a adjacent to the skin-conformal sensor 1 attached to the skin 2a was heated at a first time t1. Heating was performed until a second time t2, and heating was stopped at the second time t2. The resistance value at the first time tl was first resistance R1, and the resistance value at the second time t2 was second resistance R2. The body temperature calculation unit 53 may calculate a first body temperature value due to the first resistance R1 at the first time t1, and may calculate a second body temperature value due to the second resistance R2 at the second time t2.

Weak noise sig-n is always present in the output signal sig-t of the body temperature measurement portion 22. The reason that the noise sig-n is generated is that, since the skin-conformal sensor 1 is attached to the position through which the blood vessel 3 extends, deformation of the skin 2a due to deformation of the blood vessel 3 caused by the heart rate changes the shape of the body temperature measurement portion 22, whereby the resistance value is changed. Since the resistance value is increased as the body temperature measurement portion 22 is greatly affected by deformation of the skin 2a caused by the heart rate, it is difficult to accurately measure the body temperature at a specific time.

Referring back to FIGS. 2 and 3, a deformation prevention layer 40 is provided at the portion of the base sheet 10 at which the body temperature measurement portion 22 is formed. The skin-conformal sensor 1 may further include a deformation prevention layer 40 formed at one surface or the other surface of the base sheet 10, the deformation prevention layer being made of a material having lower elasticity than the base sheet 10 in order to limit elasticity of a part of the base sheet 10. The deformation prevention layer 40 may be formed on the base sheet 10. Since the deformation prevention layer 40 is made of a material having lower elasticity than the base sheet 10, the portion of the base sheet at which the deformation prevention layer 40 is formed may not be deformed even though the skin 2a is deformed. The deformation prevention layer 40 may be adhered to the base sheet 10 via an adhesive layer 44.

The deformation prevention layer 40 may include a temperature measurement support portion 41 formed at the portion at which the body temperature measurement portion 22 is disposed such that deformation of the skin 2a due to the heart rate is not transmitted to the body temperature measurement portion 22.

The temperature measurement support portion 41 is a part of the deformation prevention layer 40. The temperature measurement support portion 41 may be formed on the portion of the base sheet 10 at which the temperature measurement portion 20 is formed. The center of the temperature measurement support portion 41 may be aligned with the center of the temperature measurement portion 20. The temperature measurement support portion 41 may have a wider area than the temperature measurement portion 20. When the temperature measurement support portion 41 is formed at the portion of the base sheet 10 at which the temperature measurement portion 20 is formed, deformation of the temperature measurement portion 20 is minimized even though deformation of the skin 2a due to the heart rate is transmitted to the base sheet 10, since the temperature measurement support portion 41 does not expand. However, it is difficult to completely prevent deformation of the base sheet 10 even though the temperature measurement support portion 41 is formed on the base sheet 10. As a result, a part of the temperature measurement portion 20 may be deformed, and such slight deformation may appear as microscopic noise sig-n in the graph of FIG. 13.

The temperature measurement support portion 41 may be formed to cover any one of the plurality of measurement portions 20 formed in an array. The temperature measurement support portion 41 may be widely formed so as to cover one or more measurement portions 20. When the temperature measurement support portion 41 is formed so as to cover a plurality of measurement portions 20, the measurement portions 20 disposed in a region covered by the temperature measurement support portion 41 may function as temperature measurement portions 20.

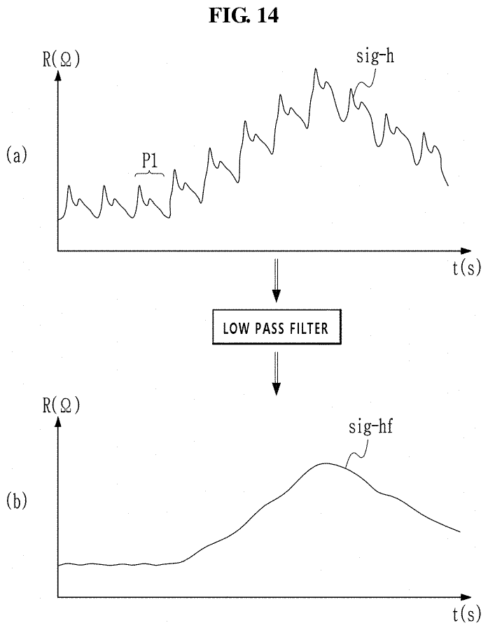

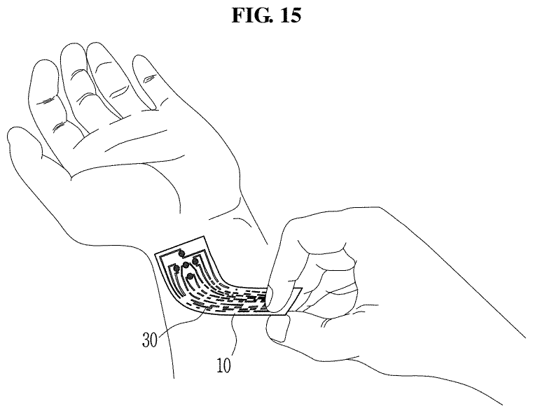

FIG. 14 is a view showing a process in which a body temperature calculation unit 53 according to an embodiment of the present invention calculates body temperature using an output signal of the heart rate measurement portion 21.

(a) of FIG. 14 is a graph showing an output signal of the heart rate measurement portion 21. The output signal of the heart rate measurement portion 21 includes a pulse according to motion of the artery 3a or the vein 3b, and the overall output signal is changed depending on a change in body temperature. If an artery pulse is present, the body temperature may be increased or decreased depending on the artery pulse, and therefore it is unsuitable to calculate body temperature in real time.

The body temperature calculation unit 53 may input the output signal of the heart rate measurement portion 21 to a low pass filter in order to acquire a filtered output signal, from which the artery pulse component has been removed, and to calculate body temperature. The body temperature calculation unit 53 may input the output signal of the heart rate measurement portion 21 to the low pass filter in order to acquire a filtered output signal, from which the artery pulse component has been removed and in which only a change in resistance value due to a change in body temperature is present. The body temperature calculation unit 53 may calculate body temperature using the filtered output signal.

An output signal including an artery pulse, an output signal including a vein pulse, and an output signal including a pulse other than the artery pulse and the vein pulse may all be input to the low pass filter. When the low pass filter removes the pulse component, the artery pulse, the vein pulse, and the ambiguous pulse component are all removed, whereby the filtered output signals have similar forms irrespective of whether the artery pulse is included.

The body temperature calculation unit 53 may average the body temperature calculated using the filtered output signal and the body temperature calculated using the output signal of the body temperature measurement portion 22 to calculate the final body temperature.

FIG. 15 is a view showing that the skin-conformal sensor 1 according to the embodiment of the present invention is separated from the skin 2a.

As shown in FIG. 15, one end of the skin-conformal sensor 1 is attached to the skin 2a. When the user pulls a part of the skin-conformal sensor 1, the portion of the skin-conformal sensor 1 may excessively expand. The base sheet 10 of the skin- conformal sensor 1 may be made of a material having elasticity similar to or higher than the elasticity of the skin 2a. When the user attaches the skin-conformal sensor 1 to the skin 2a or separates the skin-conformal sensor 1 from the skin 2a, therefore, the skin-conformal sensor 1 may excessively expand. If the skin-conformal sensor 1 expands to a damage limit or more, the electrode pattern 30 or an electrode portion may be cracked or damaged. As shown in the enlarged view of FIG. 15, the electrode pattern 30 that has expanded to the damage limit or more may be cut.

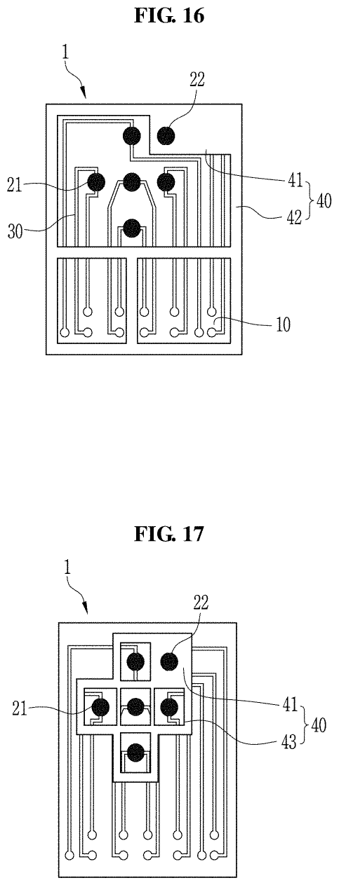

FIG. 16 is a view showing a skin-conformal sensor 1 further including a sensor shape fixing portion 42 according to an embodiment of the present invention.

The deformation prevention layer 40 may include a sensor shape fixing portion 42 formed at the edge of the base sheet 10 to inhibit expansion of the base sheet 10 to the damage limit or more. The sensor shape fixing portion 42 may prevent expansion of the skin-conformal sensor 1 to the damage limit or more, thereby preventing damage to the skin-conformal sensor 1. The sensor shape fixing portion 42 is a part of the deformation prevention layer 40. The sensor shape fixing portion 42 may be formed at the same surface as the temperature measurement support portion 41. The sensor shape fixing portion 42 may be connected to the temperature measurement support portion 41.

In FIG. 16, the sensor shape fixing portion 42 may be formed along the edge of the skin-conformal sensor 1, may be further formed in the region in which the electrode pattern 30 so as to have a “T” shape, and may be continuously connected to the temperature measurement support portion 41, which is formed in a quadrangular shape. A lower one of a limit at which the base sheet 10 expands and cannot be restored into the original shape and a limit at which the electrode pattern 30 or the measurement portion 20 expands together with the base sheet 10 and cannot be restored into the original shape may be selected as the damage limit. The sensor shape fixing portion 42 has limited elasticity and thus does not expand to more than the damage limit.

FIG. 17 is a view showing a skin-conformal sensor 1 further including a shielding portion 43 according to an embodiment of the present invention.

The deformation prevention layer 40 may include a shielding portion 43 spaced apart from the measurement portion 20, the shielding portion being constituted by one or more parts so as to surround at least a part of the measurement portion 20, the shielding portion being configured to prevent deformation of the skin 2a occurring due to a cause other than the heart rate from being transmitted to the measurement portion 20.

The shielding portion 43 is a part of the deformation prevention layer 40. The shielding portion 43 may be formed integrally with the temperature measurement support portion 41 or the sensor shape fixing portion 42. The shielding portion 43 is formed at the position spaced apart from the measurement portion 20. The shielding portion 43 may be formed so as to surround at least a part of the measurement portion 20. The shielding portion 43 may be continuously formed so as to surround the plurality of measurement portions 20. A plurality of shielding portions 42 spaced apart from each other may be formed so as to entirely surround the measurement portion 20. The shielding portion 43 may be formed so as to surround the measurement portion 20, and therefore deformation transmitted to the measurement portion 20 is minimized by the shielding portion 43 even when the skin 2a is deformed by external impact. Deformation of the skin 2a occurring due to the flow of blood by the heart rate occurs inside the shielding portion 43. Even though the shielding portion 43 is present, therefore, deformation of the measurement portion 20 due to the heart rate is not disturbed.

As described above, the skin-conformal sensor 1 is attached to the skin 2a using a skin-conformal material, whereby the skin-conformal sensor expands and contracts in response to a slight change, such as expansion of the skin 2a due to a change in volume of the blood vessel 3, and therefore it is possible to sensitively measure the heart rate. In addition, since the skin-conformal sensor is attached to the skin 2a, the position at which the sensor is attached is not changed in daily life. Furthermore, even though the user does not accurately dispose the skin-conformal sensor 1 at the position of the blood vessel 3, at least one of the plurality of measurement portions 20 disposed in an array is disposed adjacent to the blood vessel 3, whereby it is possible to measure the heart rate.

In addition, the deformation prevention layer 40 is further formed at the elastic base sheet 10, whereby deformation of the temperature measurement portion 20 is limited, damage to the base sheet 10, the measurement portion 20, and the electrode pattern 30 is prevented, and transmission of deformation of the skin 2a other than the heart rate to the measurement portion 20 is minimized.

As is apparent from the above description, according to an embodiment of the present invention, a sensor configured to be attached to the skin using a skin-conformal material is provided, whereby the position at which the sensor is attached is not changed in daily life, and it is possible to sensitively measure expansion of the skin due to a change in volume of a blood vessel, and therefore it is possible to stably and accurately measure the heart rate.

In addition, according to the embodiment of the present invention, even though a user does not accurately dispose the sensor at the position of the blood vessel, at least one of a plurality of measurement portions disposed in an array is disposed adjacent to the blood vessel, whereby it is possible to measure the heart rate.

Although the present invention has been described in detail with reference to the embodiments, the embodiments are provided to describe the present invention in detail, the present invention is not limited thereto, and the present invention can be modified or improved by a person having ordinary skill in the art to which the preset invention pertains within the technical idea of the invention.

Simple modifications and changes of the present invention are to be appreciated as being included within the scope and spirit of the invention, and the protection scope of the present invention will be defined by the accompanying claims.

What is claimed is:

1. An array type skin-conformal sensor for heart rate and body temperature measurement, the array type skin-conformal sensor comprising:
a base sheet configured to be attached to a skin in tight contact therewith, the base sheet being made of a skin-conformal material;
a measurement portion formed at one surface of the base sheet, the measurement portion being deformed by deformation of the skin, whereby resistance of the measurement portion is changed; and
an electrode pattern formed at the one surface of the base sheet, one end of the electrode pattern being connected to the measurement portion to transmit a change in resistance of the measurement portion.

2. The array type skin-conformal sensor according to claim 1, wherein the measurement portion comprises:
a plurality of heart rate measurement portions formed at the one surface of the base sheet so as to be disposed in an array; and
at least one body temperature measurement portion formed at the one surface of the base sheet.

3. The array type skin-conformal sensor according to claim 2, further comprising a deformation prevention layer formed at the one surface or the other surface of the base sheet, the deformation prevention layer being made of a material having lower elasticity than the base sheet to limit elasticity of a part of the base sheet.

4. The array type skin-conformal sensor according to claim 3, wherein the deformation prevention layer comprises a temperature measurement support portion formed at a portion at which the body temperature measurement portion is disposed such that deformation of the skin due to a heart rate is not transmitted to the body temperature measurement portion.

5. The array type skin-conformal sensor according to claim 3, wherein the deformation prevention layer comprises a sensor shape fixing portion formed at an edge of the base sheet to inhibit expansion of the base sheet to a damage limit or more.

6. The array type skin-conformal sensor according to claim 3, wherein the deformation prevention layer comprises a shielding portion spaced apart from the measurement portion, the shielding portion being constituted by one or more parts so as to surround at least a part of the measurement portion, the shielding portion being configured to prevent deformation of the skin occurring due to a cause other than a heart rate from being transmitted to the measurement portion.

7. The array type skin-conformal sensor according to claim 2, wherein the array, in which the plurality of heart rate measurement portions is disposed, is any one of a cross array, a matrix array, an oblique array, a circular array, and a decentralized array.

8. The array type skin-conformal sensor according to claim 2, wherein each of the plurality of heart rate measurement portions is formed in any one of a circular shape, a quadrangular shape, a diamond shape, a serpentine shape, and a linear shape.

9. A heart rate and body temperature measurement apparatus comprising:
a skin-conformal sensor configured to be attached to a skin, the skin-conformal sensor being configured to convert expansion and contraction of the skin caused by a heart rate into resistance change and to output the resistance change, the skin-conformal sensor also being configured to convert body temperature into resistance change and to output the resistance change; and
a reader configured to analyze the resistance change received from the skin- conformal sensor and to calculate the heart rate and a body temperature, wherein the skin-conformal sensor comprises:
a base sheet configured to be attached to the skin in tight contact therewith, the base sheet being made of a skin-conformal material;
a plurality of heart rate measurement portions formed at one surface of the base sheet so as to be disposed in an array, the plurality of heart rate measurement portions being deformed according to expansion and contraction of the skin to output resistance change;
at least one body temperature measurement portion formed at the one surface of the base sheet, the body temperature measurement portion being configured to output resistance change depending on a change in body temperature; and
an electrode pattern formed at the one surface of the base sheet, one end of the electrode pattern being connected to the heart rate measurement portions or the body temperature measurement portion to transmit a resistance change of the heart rate measurement portions or the body temperature measurement portion.

10. The heart rate and body temperature measurement apparatus according to claim 9, wherein the reader comprises:
a reception unit configured to receive the resistance change from the plurality of heart rate measurement portions and the at least one body temperature measurement portion and to generate output signals over time;
a heart rate calculation unit configured to analyze the output signals generated by the reception unit for the plurality of heart rate measurement portions and to calculate the heart rate from output signals having an identical shape to a reference waveform, excluding output signals having different shapes from the reference waveform; and
a body temperature calculation unit configured to analyze the output signal generated by the reception unit for the at least one body temperature measurement portion and to calculate a body temperature corresponding to a resistance value.

11. The heart rate and body temperature measurement apparatus according to claim 10, wherein, when one or more of the output signals generated for the plurality of heart rate measurement portions comprise an artery pulse, the heart rate calculation unit averages heart rates calculated from the output signals to calculate a final heart rate.

12. The heart rate and body temperature measurement apparatus according to claim 10, wherein the heart rate calculation unit compares an output signal comprising an artery pulse with an output signal comprising no artery pulse in real time, among waveforms corresponding to the plurality of heart rate measurement portions, when a peak having an identical shape occurs at an identical time, determines the peak to be noise, and excludes the peak from heart rate calculation.

13. The heart rate and body temperature measurement apparatus according to claim 10, wherein the body temperature calculation unit inputs the output signal of each heart rate measurement portion to a low pass filter in order to acquire a filtered output signal, from which an artery pulse component has been removed, and to calculate body temperature.
